(19) United States
(12) Patent Application Publication
(10) Pub. No.: US 2006/0216230 A1
(43) Pub. Date: Sep. 28, 2006

(54) Radioactive bone cement for treatment of tumors in bone

(75) Inventors: Joyce H Keyak, Orange, CA (US); Harry B. Skinner, Corona Del Mar, CA (US)

Correspondence Address: SHIMOKAJI & ASSOCIATES, P.C., 8911 RESEARCH DRIVE, IRVINE, CA, 92618, US

(73) Assignee: The Regents of the University of California

(21) Appl. No.: 11/388,630

(22) Filed: Mar. 24, 2006

Related U.S. Application Data
(60) Provisional application No. 60/666,041, filed on Mar. 28, 2005.

(51) Int. Cl. A61K 51/00

(52) U.S. Cl. 424/1.11

(57) ABSTRACT

Conventional internal radiation therapy has used implants, such as granules or seeds, each of which includes a radiation source for implantation within the patient. In contrast to conventional internal radiation therapy, disclosed herein is a radioactive flowable cement capable of curing to form radioactive cured cement, such that the radioactive cured cement may simultaneously provide radiation therapy, bone repair and pain management. Radioactive cement may allow for the convenient introduction of a suitable volume of radioactive material to a target site within the patient's body in a single minimally invasive procedure, as compared with the conventional need for precise implantation locations of numerous radioactive granules. In further contrast to conventional methods, radioactive cement may provide a distributed radioactive source, such that the radioactive dose rate across the target tissue (e.g., bone) can be more constant. In still further contrast to conventional methods, radioactive cement can penetrate into trabecular bone tissue prior to curing to a solid phase, thereby treating a larger volume of tissue as compared with conventional immobilized solid granules. In contrast to conventional external (beam) radiation therapy, radioactive cement may allow greater doses to the tissue to be treated, for example, by avoiding irradiation of dose-limiting structures, and enables treatment to be completed in a single procedure as opposed to multiple out-patient visits for external radiation therapy.

### CROSS-REFERENCE TO RELATED APPLICATION

This application claims benefit under 35 U.S.C. § 119(e) of U.S. Provisional Application having Ser. No. 60/666,041 filed Mar. 28, 2005, which is hereby incorporated by reference herein in its entirety.

### BACKGROUND OF THE INVENTION

The present invention relates generally to methods and compositions for treating tumors, and in particular to treating tumors in bone.

It is known in the art to use radiation therapy to kill cancer cells and shrink tumors in various organs and tissues. Radiation therapy of the prior art may be external or internal. External radiation therapy uses apparatus outside the patient's body to project a “beam” of radiation generally towards the tumor. External beam radiation therapy is typically performed by sending the patient to a radiation oncologist, and usually involves multiple visits to a hospital facility. This is inconvenient, expensive, and time consuming. In addition, in external radiation therapy, the radiation typically impacts non-target tissue, as well as the tumor to be treated, which is undesirable. This situation may be particularly problematic when the tumor is located near a dose-limiting structure, such as the spinal cord. The presence of dose-limiting structures may make external radiation therapy less effective, in that the most effective radiation dose for therapy to the target may damage the spinal cord. Also, if a patient has received prior radiation to the spinal cord, e.g., from previous radiation treatments, additional radiation to the spinal cord may not be possible without causing neurological damage. This type of situation can necessitate less aggressive radiation therapy, e.g., using sub-optimal doses.

Internal radiation therapy (brachytherapy) of the prior art has used implants, such as granules or seeds, each of which includes a radiation source, for implantation within the patient. The main benefit of brachytherapy is that greater radiation is delivered to the target and less radiation is delivered to non-target tissues. However, individual implants may not provide a uniform radiation dose rate throughout the target. In addition, numerous granular implants may be required, implantation of which may result in a lengthy and arduous procedure for both the patient and surgical team. Further, brachytherapy to treat tumors in bone has not been performed, either using granular implants or any other type of implant inside or outside the bone.

Bone cement of the prior art has been used for repairing and strengthening damaged bones, and to strengthen bone tissue. FIG. 1 is a perspective view of a solid block of conventional bone cement 10, according to the prior art. Conventional bone cement 10 may have a white or off-white (e.g., “bone”) color. Conventional bone cement 10 may be formed by combining a liquid monomer with a polymer powder to form a paste-like mixture which cures to form a solid polymer. An exemplary commercial bone cement of the prior art is known as Simplex™ (Stryker Corporation, Kalamazoo, Mich., USA), which may comprise 75% methylmethacrylate-styrene copolymer, 15% polymethylmethacrylate, and 10% barium sulfate (the latter for radiopaqueness). Conventional bone cement 10 may emit only background levels of radiation.

As can be seen, there is a need for an improved method and compositions for treating tumors in bone.

### SUMMARY OF THE INVENTION

In one aspect of the present invention, a composition comprises a biocompatible cement; and a radioactive material.

In another aspect of the present invention, a pharmaceutical composition comprises a cement; and a therapeutically effective amount of a radioactive material capable of treating a tumor.

In yet another aspect of the present invention, a method for treating a bone of a patient comprises a) forming a flowable cement mixture comprising a radioactive material; b) applying the flowable cement mixture to or in the bone; and c) curing the flowable cement mixture to form a radioactive cured cement.

In a further aspect of the present invention, a method for applying radioactive flowable cement to a bone comprises a) combining a radioactive material, a liquid monomer, and a polymer powder to form a radioactive flowable cement mixture; b) applying the radioactive flowable cement mixture to the bone; and c) allowing the radioactive flowable cement mixture to cure within the bone to form the radioactive cured cement.

In still a further aspect of the present invention, a method for treating a tumor comprises a) forming a radioactive cement mixture comprising a flowable cement and a radioactive material; b) delivering the radioactive cement mixture into or onto the bone; and c) irradiating the tumor with the delivered radioactive cement mixture.

In still another aspect of the present invention, a method for targeted irradiation of bone tissue comprises a) combining a flowable cement with a radioactive material to form a radioactive cement mixture; b) delivering the radioactive cement mixture onto or into the bone tissue; and c) irradiating the bone tissue with the delivered radioactive cement mixture.

In a further aspect of the present invention, a method for treating a tumor in the vertebral column of a patient comprises a) forming a radioactive cement mixture comprising a flowable cement and a radioactive material; b) delivering the radioactive cement mixture into or onto the vertebral column; and c) irradiating the tumor with the delivered radioactive cement mixture.

These and other features, aspects and advantages of the present invention will become better understood with reference to the following drawings, description and claims.

### BRIEF DESCRIPTION OF THE DRAWINGS

FIG. 1 is a perspective view of a solid block of conventional cement, according to the prior art;

FIG. 2A is a perspective view, and FIG. 2B is a sectional view, of a solid block of radioactive cement, according to the present invention;

FIG. 3A is a perspective view, and FIG. 3B is a sectional view, of a solid block of radioactive cement, according to another embodiment of the present invention;

FIG. 4A schematically represents a bone having a tumor therein for treatment with radioactive cement, according to another embodiment of the present invention;

FIGS. 4B-D represent stages in treating a tumor with radioactive cement, according to the present invention;

FIG. 5A is a superior view of a computer modeled structure having radioactive cement disposed therein, according to the present invention;

FIG. 5B is a lateral sectional view of the computer modeled structure of FIG. 5A;

FIG. 5C is a graph representing the radiation dose rate provided by the radioactive cement, disposed within the computer modeled structure of FIGS. 5A-B, as a function of radial distance from the radioactive cement, also according to the invention;

FIG. 6 schematically represents a series of steps involved in a method for applying radioactive cement to bone tissue, according to another embodiment of the invention; and

FIG. 7 schematically represents a series of steps involved in a method for treating a tumor, according to another embodiment of the invention.

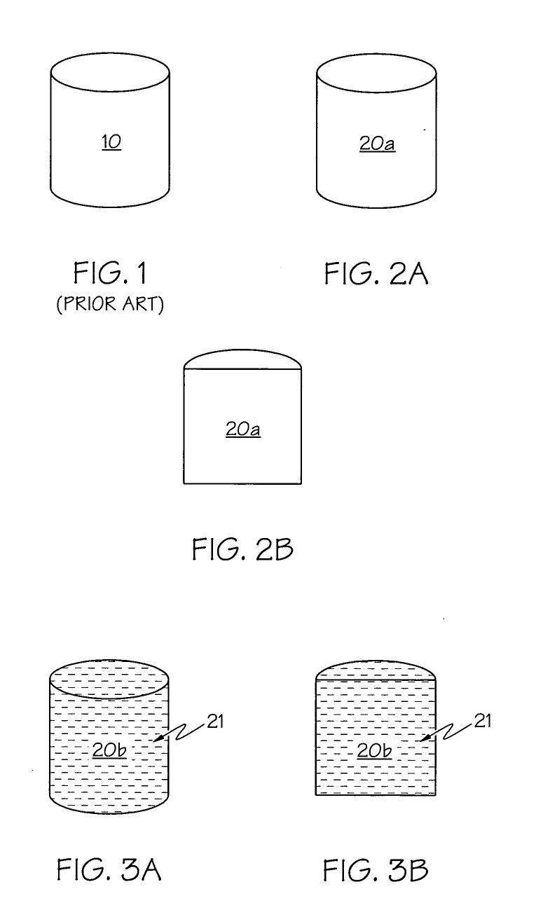

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides apparatus and methods for internal radiation therapy. The present invention may be used to provide localized irradiation of tissue in the vicinity of a tumor, as well as to simultaneously strengthen, fill, and repair bone. Moreover, the present invention may be useful in relieving pain, both by repairing bone and by irradiating the tumor cells. As a non-limiting example, the present invention may be used for treatment of tumors in bone, such as a vertebral body of the spine of a patient, wherein a radioactive cement which comprises a radioactive material may be applied to a bone. It is to be understood, however, that the present invention is not limited to treating vertebral bodies or to spinal procedures.

In contrast to conventional internal radiation therapy of the prior art, the present invention may provide a radioactive flowable cement mixture capable of curing to form radioactive cement, such that the radioactive cement may simultaneously provide radiation therapy and bone repair. Prior art internal radiation therapy (brachytherapy) for treatment of various cancers have typically used implantation of a plurality of rigid, solid granules, or “seeds,” wherein a radioactive material is included within each granule. In contrast, radioactive cement of the present invention allows for the convenient introduction of a suitable volume of radioactive material to a target site within the patient's body in a single minimally invasive procedure, as compared with the need for precise implantation locations of radioactive granules of the prior art. In further contrast to the prior art, radioactive bone cement provides a distributed radioactive source, such that the radioactive dose rate across the target tissue (e.g., bone) can be more constant. In still further contrast to the prior art, radioactive bone cement can penetrate into trabecular bone tissue prior to curing to a solid phase, thereby treating a larger volume of tissue as compared with immobilized solid granules of the prior art.

In contrast to prior art external (beam) radiation therapy, radioactive cement allows greater doses to the tissue to be treated, for example, by avoiding irradiation of dose-limiting structures, and enables treatment to be completed in a single procedure as opposed to multiple out-patient visits for external radiation therapy.

FIG. 2A is a perspective view, and FIG. 2B is a sectional view, of a solid block of radioactive cement 20a, according to the present invention. Radioactive cement 20a of the present invention may include a sufficient amount of at least one radioactive material, such that radioactive cement 20a may emit radiation above background levels. A radioactive material may be uniformly distributed within radioactive cement 20a, such that radioactive cement 20a may be uniformly radioactive throughout a given volume thereof. The radioactive material may be, as an example, a radioactive isotope known to emit high energy beta particles and low energy or no gamma radiation.

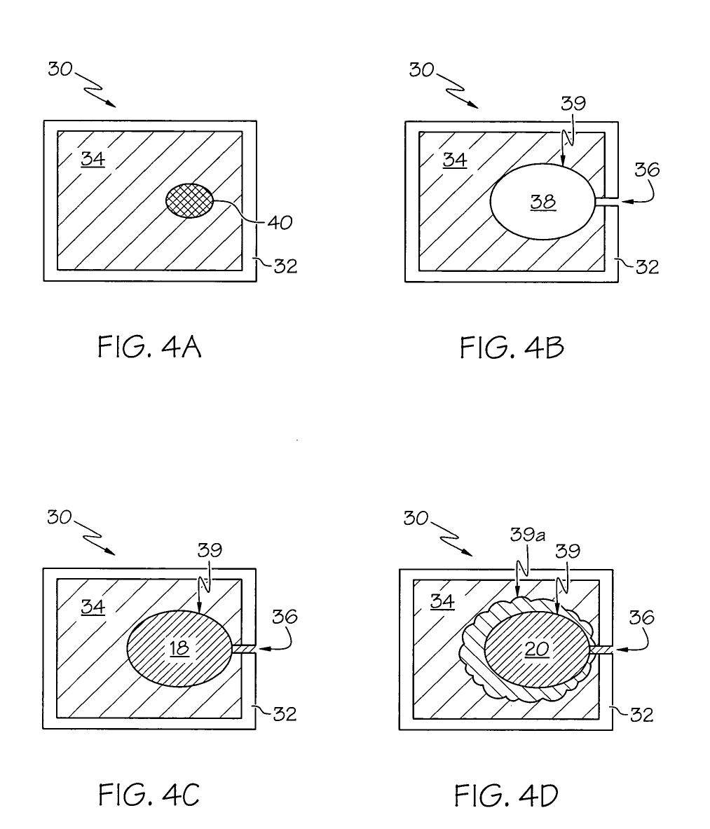

As a non-limiting example, radioactive cement 20a of the present invention may be formed by combining a radioactive isotope with a polymer powder and a liquid monomer to form a radioactive flowable cement mixture 18 (see, for example, FIG. 4C). Thereafter, flowable cement mixture 18 may be cured to provide solidified radioactive cement 20a. The time required for curing flowable cement mixture 18 to form solidified radioactive cement 20a may vary, and such time may be adjusted by varying parameters such as the temperature and composition of flowable cement mixture 18. Flowable cement mixture 18 may be a liquid, which may be viscous; or, flowable cement mixture 18 may have a paste-like consistency. Accordingly, flowable cement mixture 18 may be deformed or shaped to a particular configuration for filling one or more voids 38 (see, for example, FIG. 4B) in a bone.

Again with reference to FIGS. 2A-B, a radioactive material incorporated in radioactive cement 20a may be in the form a finely divided solid, such as a powdered radioactive material. As an example, a powdered radioactive material may be combined with polymer powder prior to mixing the polymer powder with a liquid monomer. Powdered radioactive material incorporated in radioactive cement 20a may have a mean diameter typically in the range of from about 2 to 400 microns (μm), usually from about 5 to 200 microns, and often from about 10 to 200 microns. While not being bound by theory, finely divided radioactive material may remain suspended in flowable cement mixture 18 until curing has occurred, such that the radioactive material remains uniformly distributed in solidified radioactive cement 20a.

In alternative embodiments, a liquid radioactive material may be added to the liquid monomer prior to combining the liquid monomer with a powder polymer. In other embodiments, radioactive materials may be combined with both the polymer powder and the liquid monomer prior to combining the liquid monomer with the powder polymer. As a non-limiting example, a radioactive material incorporated in radioactive cement 20a may comprise at least one radioisotope, such as phosphorus 32 (³²P), yttrium 90 (⁹⁰Y), strontium 89 (⁸⁹Sr), rhenium 186 (¹⁸⁶Re), and rhenium 188 (¹⁸⁸Re).

FIG. 3A is a perspective view, and FIG. 3B is a sectional view, of a solid block of radioactive cement 20b, according to another embodiment of the present invention. Radioactive cement 20b may have features and characteristics generally as described for radioactive cement 20a (FIGS. 2A-B). However, in the embodiment of FIGS. 3A-B, the radioactive material may be in the form of flakes 21, i.e., having a relatively high surface area to mass ratio. Such flakes 21 of radioactive material may have dimensions typically in the range of from about 0.1×1.0×1.0 mm, and usually from about 0.2×2.0×2.0 mm. Flakes 21 of radioactive material may be combined with the liquid monomer and/or polymer powder during preparation of flowable cement mixture 18, such that flakes 21 of radioactive material may remain suspended in flowable cement mixture 18 until curing has occurred, whereby flakes 21 of radioactive material may remain uniformly distributed throughout radioactive cement 20b. In alternative embodiments (not shown), the radioactive material may be in the form of short filaments, which may typically have dimensions of about 0.1×0.1×1.0 mm.

FIG. 4A schematically represents a bone 30 having a tumor 40 therein for treatment with radioactive cement 20, according to another embodiment of the present invention. The present invention is not to be limited to a particular type of bone of a human patient, domesticated animal, or the like. Instead, the present invention may be generally applicable to bones, including without limitation, long bones, short bones, and the skull. As a non-limiting example, the present invention may be applicable to various parts of the femur, such as the proximal femur; parts of the pelvis; and regions which may lack trabecular bone, such as the shafts of long bones; as well as to a vertebral body of a vertebra of the vertebral column. In FIGS. 4A-D, bone 30 to be treated may be schematically represented as a block. Bone 30 may have an outer layer of cortical bone 32, and an inner region of trabecular bone 34. Trabecular bone 34 may be encapsulated within cortical bone 32. In some situations, for example, where bone 30 may be fractured, crushed, or otherwise damaged, portions of trabecular bone 34 may be more or less exposed. Tumor 40 may be disposed entirely within trabecular bone 34 (as shown in FIG. 4A); or alternatively, tumor 40 may extend through cortical bone 32.

FIGS. 4B-D represent stages in treating a tumor in bone with radioactive cement, according to the present invention. With reference to FIG. 4B, bone 30 may be penetrated to form a port or aperture 36 in cortical bone 32, whereby trabecular bone 34 may be accessed via various surgical instruments, and the like. In some embodiments, an inflation device (not shown), such as a balloon (uninflated), may be advanced through aperture 36 and the inflation device may be inflated to form a void 38 within bone 30. In some embodiments, void 38 may be formed during a minimally invasive procedure, e.g., percutaneously, using procedures and instruments well known in the art. In some embodiments, tumor 40 may be removed prior to forming void 38. For example, tumor 40 may be surgically excised or ablated, e.g., using techniques well known in the art, such as laser ablation or electrosurgical ablation. In other embodiments, for example, where removal may be problematic due to the location of the tumor, tumor 40 may not be excised prior to, or during, the procedure. As an example, in situations where tumor 40 may be located in close proximity to the vertebral column, e.g., in a vertebral body, tumor 40 may remain in situ prior to forming void 38. In some embodiments, a void may not be formed in bone 30.

With reference to FIG. 4C, a radioactive flowable cement mixture 18 may be introduced via aperture 36 into void 38, such that the radioactive flowable cement mixture 18 may be disposed within trabecular bone 34. The flowable cement mixture 18 may be in the form of a paste or a liquid, wherein the viscosity of the paste or liquid may be varied according to the intended application. The radioactive flowable cement mixture 18 may have other features and characteristics as described hereinabove, for example, with reference to FIGS. 2A-B, 3A-B. In situations where tumor 40 may extend through cortical bone 32, flowable cement mixture 18, e.g., in the form of a high viscosity paste, may be simply placed at the location of tumor 40, or at the location whence tumor 40 was removed. Although, flowable cement mixture 18 and cement 20 are shown in FIGS. 4C-D as being disposed within trabecular bone 34, the invention is not to be so limited, but rather, in some embodiments, flowable cement mixture 18 and/or cement 20 may be disposed within both trabecular bone 34 and cortical bone 32, and in still other embodiments, flowable cement mixture 18 and/or cement 20 may be entirely restricted to cortical bone 32. Non-limiting examples of the latter situation may include treatment of a tumor in the shaft of a long bone or the pelvis.

For some bones 30 to be treated, the radioactive flowable cement mixture 18 of the present invention may be introduced into or onto bone 30 from a shielded cement cartridge (also not shown) and a shielded nozzle (also not shown) having a radioactive shield installed thereon, whereby the radioactive shield may protect operating room (OR) personnel from radiation emitted by flowable cement mixture 18. The radioactive shield may comprise, as an example, a synthetic polymer or plastic material, wherein the radioactive shield may be an effective barrier to at least beta particle emissions. Flowable cement mixture 18 may be injected into bone 30 from the shielded cement cartridge using a conventional cement gun, the latter well known in the art. Alternatively, flowable cement mixture 18 may be injected into bone 30 from a conventional cement cartridge using a shielded cement gun, the former being well known in the art. For small bones 30 to be treated, a shielded syringe may be used for introducing the radioactive flowable cement mixture 18 of the present invention into or onto bone 30.

With reference to FIG. 4D, flowable cement mixture 18 may be allowed to penetrate into trabecular bone 34 beyond the void envelope 39 to cement boundary 39a, wherein void envelope 39 may define the perimeter of void 38 (see, FIG. 4B). Accordingly, trabecular bone 34 disposed adjacent to void envelope 39 may be infused with flowable cement mixture 18, such that upon curing of flowable cement mixture 18, radioactive cement 20 may penetrate into trabecular bone 34 surrounding void envelope 39. The presence of radioactive cement 20 may allow for targeted irradiation of cancer cells within bone 30, for example, in the vicinity of void envelope 39 or cement boundary 39a.

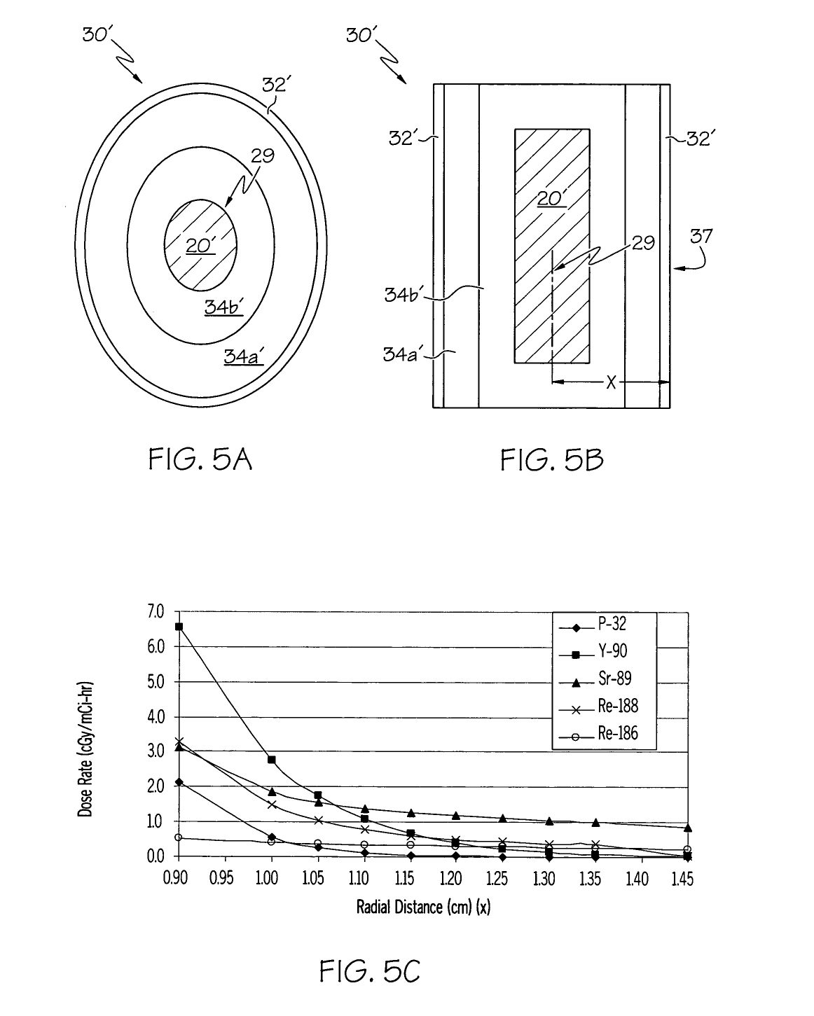

FIG. 5A is a superior view of a computer modeled structure 30′ which may be used to simulate penetration of a bone structure, such as a vertebral body of a patient, by emissions from radioactive cement 20′ disposed therein. Computer modeled structure 30′ may be substantially cylindrical, and may have a generally elliptical cross-section (see, FIGS. 5A-B). Computer modeled structure 30′ may represent bone tissue having an outer layer of cortical bone 32′, an inner layer of trabecular bone 34a′, and an innermost layer of dense trabecular bone 34b′, the latter disposed adjacent to radioactive cement 20′. Dense trabecular bone 34b′ may represent bone tissue that has been densified, or crushed, during formation of a void 38 within trabecular bone 34a′ (see, for example, FIG. 4B).

FIG. 5B is a lateral sectional view of computer modeled structure 30′ of FIG. 5A. A radial distance, x, may be shown as the distance radially outward from the center 29 of radioactive cement 20′ to the external surface 37 of computer modeled structure 30′.

FIG. 5C is a graph representing the radiation dose rate (expressed in units of cGy/mCi-hr) provided by radioactive cement 20′ as a function of radial distance, x, from the center 29 of the radioactive cement 20′, as shown in FIG. 5B. The model assumes a uniform distribution of radioactive material throughout radioactive cement 20′ (see, for example FIGS. 2A-B, 3A-B). Using a Monte Carlo Analysis, the radiation dose rate (y axis) at various locations within computer modeled structure 30′ may be provided as a function of radial distance, x, from the center 29 of radioactive cement 20′ (x axis) for different radioisotopes incorporated in radioactive cement 20′. FIG. 5C indicates that the various radioisotopes studied will deliver different dose distributions within computer modeled structure 30′. For example, ⁸⁹Sr shows good penetration through computer modeled structure 30′, and as a result, ⁸⁹Sr may deliver an almost constant dose rate with increasing radial distance, x. On the other hand, ⁹⁰Y penetrates computer modeled structure 30′ to a lesser extent, and the dose rate decreases rapidly with increasing distance from radioactive cement 20′.

With further reference to FIG. 5C, the dose rate for ¹⁸⁸Re may decrease rapidly in the outer layer of cortical bone 32′, for example, between about 1.35 cm and 1.45 cm on the x axis of FIG. 5C. The layer of cortical bone 32′ may typically be about 1 mm in thickness. While not being bound by theory, radioisotopes having emissions which may be shielded by an outer layer of cortical bone 32/32′ may be particularly useful for irradiating target tissue, while adjacent sensitive, non-target tissue, such as the spinal cord, may receive much less radiation. In this way, radiation therapy may be targeted to a target tissue, such as a tumor in bone 40 to kill cancer cells, while non-target tissue may be shielded from radiation by outer layers of bone 40. Shielding of non-target tissue from radiation may be crucial in treating tumors located adjacent to dose-limiting tissues or structures, such as the spinal cord. Radioisotopes other than those listed in FIG. 5C may also be used in conjunction with the invention.

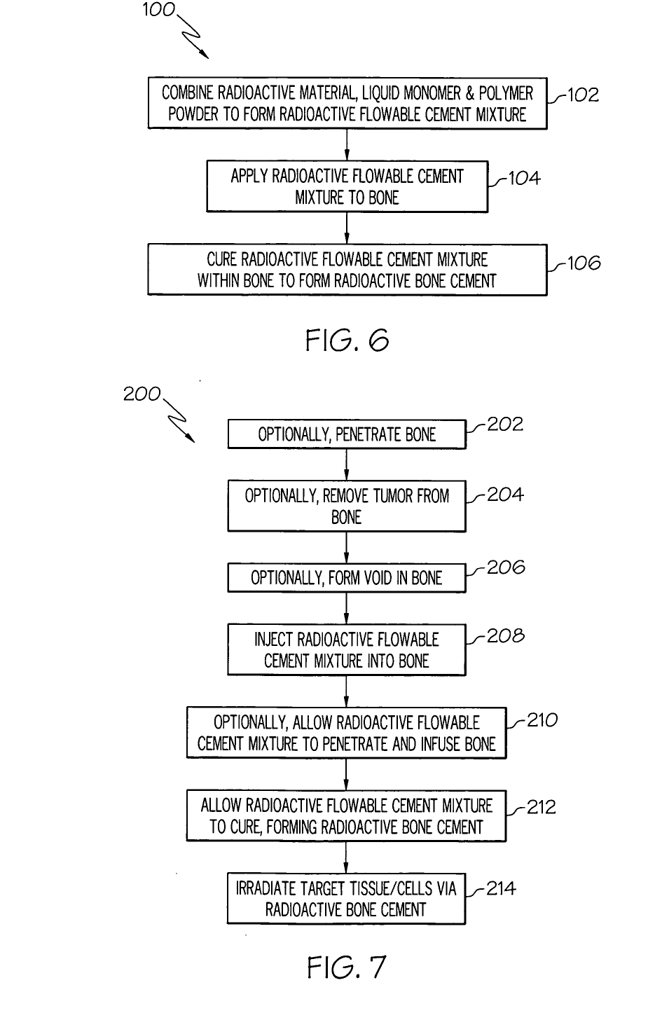

FIG. 6 schematically represents a series of steps involved in a method 100 for applying a radioactive cement to bone tissue, according to another embodiment of the invention, wherein step 102 may involve combining a radioactive material with a liquid monomer and a polymer powder to form a radioactive flowable cement mixture capable of solidifying (curing) to form a radioactive cement. The flowable cement mixture may be formed generally as described hereinabove, for example, with reference to FIGS. 2A-B, and 3A-B. As a non-limiting example, the flowable cement mixture formed in step 102 may comprise a beta-emitting radioactive material in combination with methylmethacrylate and polymethylmethacrylate. Radioisotope(s) comprising the radioactive material may be selected based on the radioisotope's emissions, the dose required in the target and the dose limits in the surrounding tissues, as well as the radioisotope's half-life, biocompatibility, availability, form (liquid vs. solid), cost and the like. A radio-opaque material, such as barium sulfate, may be included in the flowable cement mixture, to provide for fluoroscopic visualization of the flowable cement mixture, and of the resultant radioactive cement, during a bone treatment procedure of the present invention.

Step 104 may involve applying the flowable cement mixture to bone tissue. As a non-limiting example, the flowable cement mixture may be applied to a void within trabecular bone (see, for example, FIG. 4C). During preparation of the flowable cement mixture, personnel may be shielded from radiation emitted by the flowable cement mixture, or its constituents, for example, using a suitable shielding device or barrier. Apparatus for shielding personnel from exposure to radioactive materials are well known in the art.

Step 106 may involve curing the flowable cement mixture, or allowing the flowable cement mixture to solidify within or on a bone, to form a solidified radioactive cement, wherein the radioactive cement may be capable of irradiating a tumor or other target tissue within, or adjacent to, the bone to which the flowable cement mixture may have been applied in step 104.

FIG. 7 schematically represents a series of steps involved in a method 200 for treating a tumor in or adjacent a bone, according to another embodiment of the invention. Optional step 202 may involve penetrating the bone having the tumor, so as to form an aperture in the outer layer of cortical bone, whereby the inner trabecular bone and/or the tumor may be accessed. The aperture in the cortical bone may be formed using conventional procedures and instruments, well known in the prior art (see, for example, FIG. 4B). In some embodiments or instances, the bone to be treated may be damaged to an extent that at least one of the tumor to be treated and the trabecular bone may be exposed due to fracture or disintegration of the layer of cortical bone. In such instances, step 202 may be omitted. In still other embodiments, treatment may be limited to cortical bone, such that trabecular bone may not be accessed.

Optional step 204 may involve removing at least a portion of the tumor from the bone to be treated. The tumor may be surgically excised or ablated, generally as described hereinabove, for example, with reference to FIG. 4B, using prior art procedures and apparatus. In situations where the location of the tumor may cause removal of the tumor to be problematic, for example, for tumors located in the spine (vertebral column), step 204 may be omitted.

Step 206 may involve forming a void in the bone to be treated. The void may be formed in trabecular bone using an inflation device, such as a balloon, and a liquid or gaseous inflation medium. Procedures for forming voids in bone tissue are well known in the art. In some instances, a bone to be treated may have one or more pre-existing voids, for example, due to various forms of bone damage, excision of a tumor, or the like, in which event step 206 may be omitted.

Step 208 may involve injecting, placing, or otherwise delivering, a radioactive flowable cement mixture into or onto the bone to be treated. The flowable cement mixture may be injected into one or more voids in the bone to be treated. Alternatively, the flowable cement mixture may be injected into, or otherwise applied to, trabecular bone without forming a void therein. The flowable cement mixture to be injected may have characteristics and features as described hereinabove, e.g., with reference to FIGS. 2A-3B. The flowable cement mixture may be prepared generally as described hereinabove, e.g., with reference to FIGS. 2A-3B, and FIG. 6. During injection of the flowable cement mixture into the bone to be treated, OR personnel may be shielded from radiation emitted by the flowable cement mixture or its constituents, for example, using a shielded cement cartridge, and/or a shielded cement gun. The flowable cement mixture may be injected under fluoroscopic (radiographic) control to enable precise placement thereof.

Optional step 210 may involve allowing the flowable cement mixture to penetrate into trabecular bone, such that the trabecular bone surrounding the void may be infused with the flowable cement mixture. As an example, the flowable cement mixture injected into the bone may be a low viscosity cement mixture, such that the distance to which the flowable cement mixture may penetrate into the trabecular bone may be increased.

Step 214 may involve irradiating a tumor or other target tissue via the radioactive cement disposed within the bone adjacent to the target tissue. As a non-limiting example, the radioactive cement may comprise a beta emitting radioisotope, and the radioactive cement may be capable of delivering a therapeutically effective dose rate of beta particle emissions to a tumor or other target tissue so as to kill cancer cells of the target tissue. The radioactive cement may comprise at least one radioisotope, such as phosphorus 32 (³²P), yttrium 90 (⁹⁰Y), strontium 89 (⁸⁹Sr), rhenium 186 (¹⁸⁶Re), and rhenium 188 (¹⁸⁸Re).

While the invention has been described primarily with respect to cement containing polymethylmethacrylate, the present invention is not meant to be limited in this respect. For example, any conventional cement or bone joining medium may be used to incorporate a radioactive material according to the compositions and methods of the present invention. Moreover, while the above description makes reference to cement or bone cement, any flowable biocompatible cement may be used in the embodiments of the present invention.

Although the invention has been described primarily with respect to treating tumors in bone tissue, radioactive cement of the invention may also find applications in treating soft tissue and/or soft tissue tumors.

It should be understood, of course, that the foregoing relates to exemplary embodiments of the invention and that modifications may be made without departing from the spirit and scope of the invention as set forth in the following claims.

We claim:

1. A composition, comprising:
a biocompatible cement; and
a radioactive material.

2. The composition according to claim 1, wherein the cement comprises a bone cement comprising a polymer powder and a liquid monomer.

3. The composition according to claim 1, wherein the cement comprises polymethylmethacrylate and methylmethacrylate-styrene copolymer.

4. The composition according to claim 3, wherein the cement further comprises a radio-opaque additive.

5. The composition according to claim 1, wherein the radioactive material comprises at least one radioactive material selected from the group consisting of phosphorus 32 (³²P), yttrium 90 (⁹⁰Y), strontium 89 (⁸⁹Sr), rhenium 186 (¹⁸⁶Re), and rhenium 188 (¹⁸⁸Re).

6. The composition according to claim 1, wherein the cement is cured and the radioactive material is uniformly distributed throughout the cement.

7. The composition according to claim 1, wherein the radioactive material is in the form of filaments or flakes.

8. The composition according to claim 1, wherein the radioactive material is in the form of at least one of a liquid and a powder.

9. A pharmaceutical composition comprising:
a cement; and
a therapeutically effective amount of a radioactive material capable of treating a tumor in a bone.

10. The pharmaceutical composition according to claim 9, wherein the radioactive material comprises at least one radioactive material selected from the group consisting of phosphorus 32 (³²P), yttrium 90 (⁹⁰Y), strontium 89 (⁸⁹Sr), rhenium 186 (¹⁸⁶Re), and rhenium 188 (¹⁸⁸Re).

11. A method for treating a bone of a patient, comprising:
a) forming a flowable cement mixture comprising a radioactive material;
b) applying said flowable cement mixture onto or in the bone; and
c) curing the flowable cement mixture to form a radioactive cured cement.

12. The method according to claim 11, wherein the flowable cement mixture is applied onto the bone.

13. The method according to claim 11, wherein the flowable cement mixture is applied within the bone.

14. The method according to claim 11, further comprising penetrating a cortical bone portion of the bone to access a trabecular bone portion of the bone.

15. The method according to claim 11, further comprising forming a void within the bone.

16. The method according to claim 15, wherein the flowable cement mixture is applied in the void.

17. A method for applying radioactive cement to a bone, comprising:
a) combining a radioactive material, a liquid monomer, and a polymer powder to form a radioactive flowable cement mixture;
b) applying said radioactive flowable cement mixture to said bone; and
c) allowing said radioactive flowable cement mixture to cure within said bone to form said radioactive cured cement.

18. The method according to claim 17, further comprising:
penetrating a cortical bone portion of the bone to access a trabecular bone portion of the bone; and
forming a void within the bone.

19. The method according to claim 18, further comprising:
allowing the radioactive cement mixture to penetrate into trabecular bone of the trabecular bone portion; and
allowing the radioactive cement mixture to cure to form a radioactive cured cement infused within the trabecular bone.

20. A method for treating a tumor, comprising:
a) forming a radioactive cement mixture comprising a flowable cement and a radioactive material;
b) delivering the radioactive cement mixture into or onto bone; and
c) irradiating the tumor with the delivered radioactive cement mixture.

21. The method according to claim 20, further comprising penetrating a cortical bone portion of the bone to access a trabecular bone portion of the bone.

22. The method according to claim 21, further comprising forming a void within the trabecular bone portion.

23. The method according to claim 22, wherein the radioactive cement mixture is delivered in the void.

24. The method according to claim 20, further comprising:
delivering the radioactive cement mixture into the bone; and
allowing the radioactive cement mixture to penetrate into trabecular bone

25. The method according to claim 23, further comprising allowing the radioactive cement mixture to penetrate into trabecular bone of the trabecular bone portion.

26. The method according to claim 25, further comprising allowing the radioactive cement mixture to cure to form a radioactive bone cement infused within the trabecular bone.

27. The method according to claim 20, further comprising removing at least a portion of the tumor from the bone.

28. The method according to claim 27, wherein the radioactive cement mixture is delivered to a void formed from the step of removing at least a portion of the tumor from the bone.

29. The method according to claim 20, wherein the delivered radioactive cement mixture emits a therapeutically effective dose rate of radiation sufficient to kill cancer cells.

30. The method according to claim 20, wherein the flowable cement comprises a liquid monomer and a polymer powder.

31. The method according to claim 29, wherein the flowable cement further comprises a radio-opaque material.

32. A method for targeted irradiation of bone tissue, comprising:
a) combining a flowable cement with a radioactive material to form a radioactive cement mixture;
b) delivering the radioactive cement mixture onto or into the bone tissue; and
c) irradiating the bone tissue with the delivered radioactive cement mixture.

33. The method according to claim 32, further comprising penetrating a cortical bone portion of the bone tissue to access a trabecular bone portion of the bone tissue.

34. The method according to claim 33, further comprising forming a void within the trabecular bone portion of the bone tissue.

35. The method according to claim 34, wherein the radioactive cement mixture is delivered in the void.

36. The method according to claim 35, further comprising allowing the radioactive cement mixture to penetrate into trabecular bone of the trabecular bone portion.

37. The method according to claim 36, further comprising allowing the radioactive cement mixture to cure to form a radioactive cured cement infused within the trabecular bone.

38. A method for treating a tumor in the vertebral column of a patient, comprising:
a) forming a radioactive cement mixture comprising a flowable cement and a radioactive material;
b) delivering the radioactive cement mixture into or onto the vertebral column; and
c) irradiating the tumor with the delivered radioactive cement mixture.

39. The method according to claim 38, further comprising forming a void within a vertebral body.

40. The method according to claim 39, wherein the radioactive cement mixture is delivered in the void.

41. The method according to claim 40, further comprising allowing the radioactive cement mixture to penetrate into trabecular bone of the vertebral body.

42. The method according to claim 41, further comprising allowing the radioactive cement mixture to cure to form a radioactive cement infused within the trabecular bone of the vertebral body.

43. The method according to claim 38, further comprising:
delivering the radioactive cement mixture into the vertebral column; and
allowing the radioactive cement mixture to penetrate into trabecular bone.

44. The method according to claim 38, further comprising removing at least a portion of the tumor from the vertebral column.

45. The method according to claim 44, wherein the radioactive cement mixture is delivered to a void formed from the step of removing at least a portion of the tumor from the vertebral column.
